(19) United States
(12) Patent Application Publication
(10) Pub. No.: US 2011/0015314 A1
(43) Pub. Date: Jan. 20, 2011

(54) ULTRAVIOLET ABSORBENT AND PRODUCTION METHOD OF THE SAME

(75) Inventors: Youichiro Takeshima, Kanagawa (JP); Keizo Kimura, Kanagawa (JP)

Correspondence Address: BUCHANAN, INGERSOLL & ROONEY PC, POST OFFICE BOX 1404, ALEXANDRIA, VA, 22313-1404, US

(73) Assignee: FUJIFILM Corporation, Minato-ku (JP)

(21) Appl. No.: 12/934,676

(22) PCT Filed: Mar. 30, 2009
(86) PCT No.: PCT/JP2009/056557

§ 371 Date: Sep. 27, 2010

(30) Foreign Application Priority Data
Mar. 31, 2008 (JP) 2008-091833

(51) Int. Cl. C08K 5/357; C07D 265/22

(52) U.S. Cl. 524/87; 544/92

(57) ABSTRACT

An ultraviolet absorbent containing a compound represented by the following formula (I), having an aluminum ion in a concentration of less than 2 ppm, and an iron ion in a concentration of less than 2 ppm.

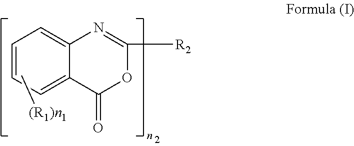

wherein R₁ represents a substituent; n₁ represents an integer of 0 to 4; R₂ represents an n₂-valent substituent or a linking group; and n₂ represents an integer of 1 to 4.

### TECHNICAL FIELD

The present invention relates to an ultraviolet absorbent and a production method of the same. More particularly, the present invention relates to a benzoxazinone-based ultraviolet absorbent and a production method of the same.

### BACKGROUND ART

Benzotriazole-based compounds, benzophenone-based compounds, salicylic acid-based compounds, triazine-based compounds, or the like have been used as an ultraviolet absorbent for a thermoplastic polymer. These ultraviolet absorbents are generally problematic in terms of insufficient ultraviolet-shielding capability, poor resistance to heat, easy coloration, poor fastness, and the like.

Benzoxadinone-based compounds are proposed as an ultraviolet absorbent that addresses these problems (see, for example, JP-B-62-5944 (“JP-B” means examined Japanese patent publication) or JP-B-62-31027). In order to utilize the characteristics of the benzoxadinone-based compound, a production method of the compound in which the sodium content is reduced is proposed from viewpoints of not only low coloration suitable for applications to articles with high degree of transparency, but also prevention of polymer from deterioration (see, for example, JP-T-2005-507006 (“JP-T” means published Japanese translation of PCT application)). Further, in order to improve both storability and heat resistance of the benzoxadinone-based compound itself and to obtain a molded article with an inherent transparency in such a manner that when the benzoxadinone-based compound is added to a thermoplastic polymer and then kneaded, both workability and working environment in the kneading process and the molding process of the kneaded mixture are not deteriorated, a production method of the compound in which both acid value and chloride ion concentration are controlled in specific ranges respectively is proposed (see, for example, Japanese Patent No. 3874407).

### DISCLOSURE OF INVENTION

The present invention is to contemplate for providing a benzoxadinone-based ultraviolet absorbent that has a low content of metal ions and that is able to reduce deterioration of a thermoplastic polymer, when the benzoxadinone-based ultraviolet absorbent is added to the thermoplastic polymer and then kneaded. Further, the present invention is to contemplate for providing a method of producing the above-described benzoxadinone-based ultraviolet absorbent.

The present invention provides the following means:

[1] An ultraviolet absorbent comprising a compound represented by the following formula (I), having an aluminum ion in a concentration of less than 2 ppm (not including 0 ppm), and an iron ion in a concentration of less than 2 ppm (not including 0 ppm).

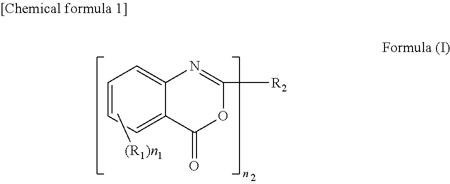

wherein R₁ represents a substituent; n₁ represents an integer of 0 to 4; R₂ represents an n₂-valent substituent or a linking group; and n₂ represents an integer of 1 to 4.

[2] The ultraviolet absorbent as described in the above item [1], wherein the aluminum ion concentration is less than 1 ppm (not including 0 ppm), and the iron ion concentration is less than 1 ppm (not including 0 ppm).
[3] The ultraviolet absorbent as described in the above item [1] or [2], wherein the aluminum ion concentration is less than 0.5 ppm (not including 0 ppm), and the iron ion concentration is less than 0.5 ppm (not including 0 ppm).
[4] The ultraviolet absorbent described in any one of [1] to [3], further having a calcium ion in a concentration of less than 1 ppm (not including 0 ppm).
[5] A method of producing the ultraviolet absorbent as described in any one of the above items [1] to [4], comprising:

a process A in which an anthranilic acid compound is allowed to react with a carboxylic acid halide in the absence of a base, without isolating an amide intermediate compound represented by the following formula (II).

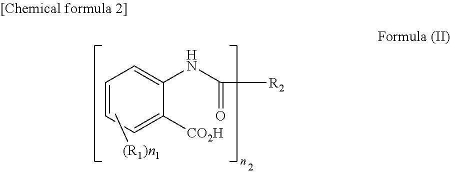

wherein R₁ represents a substituent; n₁ represents an integer of 0 to 4; R₂ represents an n₂-valent substituent or a linking group; and n₂ represents an integer of 1 to 4.

[6] The method as described in the above item [5], wherein at least one of reaction solvents used in the process A has a number of donor of 10 or more (preferably 10 or more and 50 or less).
[7] The method as described in the above item [5] or [6], wherein no protic solvent is used in the process A.
[8] The method as described in any one of the above items [5] to [7], wherein the temperature at the process A is 50° C. or lower (preferably −30° C. or higher and 50° C. or lower).
[9] A polymer composition, comprising the ultraviolet absorbent as described in any one of the above items [1] to [4], and a polymer substance.
[10] The polymer composition described in [9], wherein the polymer composition is a film.
[11] The polymer composition as described in the above item [9] or [10], wherein the polymer substance is a polyester.
[12] The polymer composition as described in any one of the above items [9] to [11], wherein the polymer substance is a polyethylene terephthalate.

### ADVANTAGEOUS EFFECTS OF INVENTION

The benzoxadinone-based ultraviolet absorbent of the present invention makes it possible to reduce deterioration of a thermoplastic polymer when used in the form of the ultraviolet absorbent kneaded in the thermoplastic polymer. Further, according to the method of the present invention, it is possible to produce a high-quality benzoxadinone-based ultraviolet absorbent having a low content of metal ions.

Other and further features and advantages of the invention will appear more fully from the following description.

### BEST MODE FOR CARRYING OUT INVENTION

Embodiments of the present invention will be explained hereinbelow.

In this specification, first, the aliphatic group means alkyl groups, substituted alkyl groups, alkenyl groups, substituted alkenyl groups, alkynyl groups, substituted alkynyl groups, aralkyl groups and substituted aralkyl groups. The alkyl groups may be branched or may form a ring. The number of carbon atoms of the alkyl group is preferably 1 to 20 and more preferably 1 to 18. The alkyl moiety of the substituted alkyl group is the same as the above alkyl group. The alkenyl moiety of the substituted alkenyl group is the same as the above alkenyl group. The alkenyl group may be branched or may form a ring. The number of carbon atoms of the alkenyl group is preferably 2 to 20 and more preferably 2 to 18. The alkenyl moiety of the substituted alkenyl group is the same as the above alkenyl group. The alkynyl group may be branched or may form a ring. The number of carbon atoms of the alkynyl group is preferably 2 to 20 and more preferably 2 to 18. The alkynyl moiety of the substituted alkynyl group is the same as the above alkynyl group. The alkyl group of the aralkyl group and substituted aralkyl group is the same as the above alkyl group. The aryl moiety of the aralkyl group and the substituted aralkyl group is the same as the following aryl group.

Examples of the substituent in the alkyl moiety of the substituted alkyl group, substituted alkenyl groups, substituted alkynyl groups and substituted aralkyl groups include a halogen atom (e.g., chlorine, bromine, iodine atom), an alkyl group [which means a linear, branched or cyclic substituted or unsubstituted alkyl group and which includes an alkyl group (preferably an alkyl group having 1 to 30 carbon atoms, e.g., methyl, ethyl, n-propyl, isopropyl, tert-butyl, n-octyl, eicosyl, 2-chloroethyl, 2-cyanoethyl, 2-ethylhexyl), a cycloalkyl group (preferably a substituted or unsubstituted cycloalkyl group having 3 to 30 carbon atoms, e.g., cyclohexyl, cyclopentyl, 4-n-dodecyl-cyclohexyl), a bicycloalkyl group (preferably a substituted or unsubstituted bicycloalkyl group having 5 to 30 carbon atoms, namely, a monovalent group resultant from removing one hydrogen atom of a bicycloalkane having 5 to 30 carbon atoms, e.g., bicyclo[1,2,2]heptan-2-yl, bicyclo[2,2,2]octan-3-yl), and a group having many cyclic structures, such as tricyclo-structure; the alkyl group in the substituents described below (for example, an alkyl group in an alkylthio group) means an alkyl group having such a concept],

an alkenyl group [which means a linear, branched or cyclic substituted or unsubstituted alkenyl group and which includes an alkenyl group (preferably a substituted or unsubstituted alkenyl group having 2 to 30 carbon atoms, e.g., vinyl, allyl, prenyl, geranyl, oleyl), a cycloalkenyl group (preferably a substituted or unsubstituted cycloalkenyl group having 3 to 30 carbon atoms, namely, a monovalent group resultant from removing one hydrogen atom of a cycloalkene having 3 to 30 carbon atoms, e.g., 2-cyclopenten-1-yl, 2-cyclohexen-1-yl), and a bicycloalkenyl group (a substituted or unsubstituted bicycloalkenyl group, preferably a substituted or unsubstituted bicycloalkenyl group having 5 to 30 carbon atoms, namely, a monovalent group resultant from removing one hydrogen atom of a bicycloalkene having one double bond, e.g., bicyclo[2,2,1]hept-2-en-1-yl, bicyclo[2,2,2]oct-2-en-4-yl)], an alkynyl group (preferably a substituted or unsubstituted alkynyl group having 2 to 30 carbon atoms, e.g., ethynyl, propargyl, trimethylsilylethynyl),
an aryl group (preferably a substituted or unsubstituted aryl group having 6 to 30 carbon atoms, e.g., phenyl, p-tolyl, naphthyl, m-chlorophenyl, o-hexadecanoylaminophenyl), a heterocyclic group (preferably a monovalent group resultant from removing one hydrogen atom of a 5- or 6-membered substituted or unsubstituted aromatic or non-aromatic heterocyclic compound, more preferably a 5- or 6-membered aromatic heterocyclic group having 3 to 30 carbon atoms, e.g., 2-furyl, 2-thienyl, 2-pyrimidinyl, 2-benzothiazolyl), a cyano group, a hydroxyl group, a nitro group, a carboxyl group, an alkoxy group (preferably a substituted or unsubstituted alkoxy group having 1 to 30 carbon atoms, e.g., methoxy, ethoxy, isopropoxy, tert-butoxy, n-octyloxy, 2-methoxyethoxy), an aryloxy group (preferably a substituted or unsubstituted aryloxy group having 6 to 30 carbon atoms, e.g., phenoxy, 2-methylphenoxy, 4-tert-butylphenoxy, 3-nitrophenoxy, 2-tetradecanoylaminophenoxy),
a silyloxy group (preferably a silyloxy group having 3 to 20 carbon atoms, e.g., trimethylsilyloxy, tert-butyldimethylsilyloxy), a heterocyclic oxy group (preferably a substituted or unsubstituted heterocyclic oxy group having 2 to 30 carbon atoms, e.g., 1-phenyltetrazol-5-oxy, 2-tetrahydropyranyloxy), an acyloxy group (preferably a formyloxy group, a substituted or unsubstituted alkylcarbonyloxy group having 2 to 30 carbon atoms or a substituted or unsubstituted arylcarbonyloxy group having 6 to 30 carbon atoms, e.g., formyloxy, acetyloxy, pivaloyloxy, stearoyloxy, benzoyloxy, p-methoxyphenylcarbonyloxy), a carbamoyloxy group (preferably a substituted or unsubstituted carbamoyloxy group having 1 to 30 carbon atoms, e.g., N,N-dimethylcarbamoyloxy, N,N-diethylcarbamoyloxy, morpholinocarbonyloxy, N,N-di-n-octylaminocarbonyloxy, N-n-octylcarbamoyloxy),
an alkoxycarbonyloxy group (preferably a substituted or unsubstituted alkoxycarbonyloxy group having 2 to 30 carbon atoms, e.g., methoxycarbonyloxy, ethoxycarbonyloxy, tert-butoxycarbonyloxy, n-octyloxycarbonyloxy), an aryloxycarbonyloxy group (preferably a substituted or unsubstituted aryloxycarbonyloxy group having 7 to 30 carbon atoms, e.g., phenoxycarbonyloxy, p-methoxyphenoxycarbonyloxy, p-n-hexadecyloxyphenoxycarbonyloxy), an amino group (preferably an amino group, a substituted or unsubstituted alkylamino group having 1 to 30 carbon atoms or a substituted or unsubstituted anilino group having 6 to 30 carbon atoms, e.g., amino, methylamino, dimethylamino, anilino, N-methyl-anilino, diphenylamino), an acylamino group (preferably a formylamino group, a substituted or unsubstituted alkylcarbonylamino group having 1 to 30 carbon atoms or a substituted or unsubstituted arylcarbonylamino group having 6 to 30 carbon atoms, e.g., formylamino, acetylamino, pivaloylamino, lauroylamino, benzoylamino, 3,4,5-tri-n-octyloxyphenylcarbonylamino),
an aminocarbonylamino group (preferably a substituted or unsubstituted aminocarbonylamino group having 1 to 30 carbon atoms, e.g., carbamoylamino, N,N-dimethylaminocarbonylamino, N,N-diethylaminocarbonylamino, morpholinocarbonylamino), an alkoxycarbonylamino group (preferably a substituted or unsubstituted alkoxycarbonylamino group having 2 to 30 carbon atoms, e.g., methoxycarbonylamino, ethoxycarbonylamino, tert-butoxycarbonylamino, n-octadecyloxycarbonylamino, N-methyl-methoxycarbonylamino), an aryloxycarbonylamino group (preferably a substituted or unsubstituted aryloxycarbonylamino group having 7 to 30 carbon atoms, e.g., phenoxycarbonylamino, p-chlorophenoxycarbonylamino, m-(n-octyloxy)phenoxycarbonylamino), a sulfamoylamino group (preferably a substituted or unsubstituted sulfamoylamino group having 0 to 30 carbon atoms, e.g., sulfamoylamino, N,N-dimethylaminosulfonylamino, N-n-octylaminosulfonylamino),
an alkyl- or aryl-sulfonylamino group (preferably a substituted or unsubstituted alkylsulfonylamino group having 1 to 30 carbon atoms or a substituted or unsubstituted arylsulfonylamino group having 6 to 30 carbon atoms, e.g., methylsulfonylamino, butylsulfonylamino, phenylsulfonylamino, 2,3,5-trichlorophenylsulfonylamino, p-methylphenylsulfonylamino), a mercapto group, an alkylthio group (preferably a substituted or unsubstituted alkylthio group having 1 to 30 carbon atoms, e.g., methylthio, ethylthio, n-hexadecylthio), an arylthio group (preferably a substituted or unsubstituted arylthio group having 6 to 30 carbon atoms, e.g., phenylthio, p-chlorophenylthio, m-methoxyphenylthio), a heterocyclic thio group (preferably a substituted or unsubstituted heterocyclic thio group having 2 to 30 carbon atoms, e.g., 2-benzothiazolylthio, 1-phenyltetrazol-5-ylthio),
a sulfamoyl group (preferably a substituted or unsubstituted sulfamoyl group having 0 to 30 carbon atoms, e.g., N-ethylsulfamoyl, N-(3-dodecyloxypropyl)sulfamoyl, N,N-dimethylsulfamoyl, N-acetylsulfamoyl, N-benzoylsulfamoyl, N—(N′-phenylcarbamoyl)sulfamoyl), a sulfo group, an alkyl- or aryl-sulfinyl group (preferably a substituted or unsubstituted alkylsulfinyl group having 1 to 30 carbon atoms or a substituted or unsubstituted arylsulfinyl group having 6 to 30 carbon atoms, e.g., methylsulfinyl, ethylsulfinyl, phenylsulfinyl, p-methylphenylsulfinyl), an alkyl- or aryl-sulfonyl group (preferably a substituted or unsubstituted alkylsulfonyl group having 1 to 30 carbon atoms or a substituted or unsubstituted arylsulfonyl group having 6 to 30 carbon atoms, e.g., methylsulfonyl, ethylsulfonyl, phenylsulfonyl, p-methylphenylsulfonyl),
an acyl group (preferably a formyl group, a substituted or unsubstituted alkylcarbonyl group having 2 to 30 carbon atoms, a substituted or unsubstituted arylcarbonyl group having 7 to 30 carbon atoms or a substituted or unsubstituted heterocyclic carbonyl group having 4 to 30 carbon atoms and being bonded to a carbonyl group through a carbon atom, e.g., acetyl, pivaloyl, 2-chloroacetyl, stearoyl, benzoyl, p-n-octyloxyphenylcarbonyl, 2-pyridylcarbonyl, 2-furylcarbonyl), an aryloxycarbonyl group (preferably a substituted or unsubstituted aryloxycarbonyl group having 7 to 30 carbon atoms, e.g., phenoxycarbonyl, o-chlorophenoxycarbonyl, m-nitrophenoxycarbonyl, p-tert-butylphenoxycarbonyl), an alkoxycarbonyl group (preferably a substituted or unsubstituted alkoxycarbonyl group having 2 to 30 carbon atoms, e.g., methoxycarbonyl, ethoxycarbonyl, tert-butoxycarbonyl, n-octadecyloxycarbonyl), a carbamoyl group (preferably a substituted or unsubstituted carbamoyl group having 1 to 30 carbon atoms, e.g., carbamoyl, N-methylcarbamoyl, N,N-dimethylcarbamoyl, N,N-di-n-octylcarbamoyl, N-(methylsulfonyl)-carbamoyl),
an aryl- or heterocyclic-azo group (preferably a substituted or unsubstituted arylazo group having 6 to 30 carbon atoms or a substituted or unsubstituted heterocyclic-azo group having 3 to 30 carbon atoms, e.g., phenylazo, p-chlorophenylazo, 5-ethylthio-1,3,4-thiadiazol-2-ylazo), an imido group (preferably N-succinimido, N-phthalimido), a phosphino group (preferably a substituted or unsubstituted phosphino group having 2 to 30 carbon atoms, e.g., dimethylphosphino, diphenylphosphino, methylphenoxyphosphino), a phosphinyl group (preferably a substituted or unsubstituted phosphinyl group having 2 to 30 carbon atoms, e.g., phosphinyl, dioctyloxyphosphinyl, diethoxyphosphinyl), a phosphinyloxy group (preferably a substituted or unsubstituted phosphinyloxy group having 2 to 30 carbon atoms, e.g., diphenoxyphosphinyloxy, dioctyloxyphosphinyloxy), a phosphinylamino group (preferably a substituted or unsubstituted phosphinylamino group having 2 to 30 carbon atoms, e.g., dimethoxyphosphinylamino, dimethylaminophosphinylamino), or a silyl group (preferably a substituted or unsubstituted silyl group having 3 to 30 carbon atoms, e.g., trimethylsilyl, tert-butyldimethylsilyl, phenyldimethylsilyl).

Among the above functional groups, those having a hydrogen atom may be further substituted with the above group at the position from which the hydrogen atom is removed. Examples of such functional groups include an alkylcarbonylaminosulfonyl group, arylcarbonylaminosulfonyl group, alkylsulfonylaminocarbonyl group and arylsulfonylaminocarbonyl group. Specific examples of these groups include methylsulfonylaminocarbonyl, p-methylphenylsulfonylaminocarbonyl, acetylaminosulfonyl and benzoylaminosulfonyl group.

Examples of the substituent of the aryl moiety of the substituted aralkyl group are the same as substituents of the following substituted aryl group.

The aromatic group in this specification means an aryl group and a substituted aryl group. Further, these aromatic groups may be condensed with aliphatic rings, other aromatic rings or hetero rings. The number of carbon atoms of the aromatic group is preferably 6 to 40, more preferably 6 to 30 and still more preferably 6 to 20. Among these groups, the aryl group is preferably a phenyl or naphthyl group and particularly preferably a phenyl group.

The aryl moiety of the substituted aryl group is the same as the above aryl group. Examples of the substituent of the substituted aryl group are the same as those listed above as the substituent of the alkyl moiety of the previous substituted alkyl group, substituted alkenyl group, substituted alkynyl group and substituted aralkyl group.

In the present specification, the heterocyclic group preferably contains a 5- or 6-membered saturated or unsaturated heterocycle. An aliphatic ring, an aromatic ring or another heterocycle may be fused with the heterocycle. Examples of a heteroatom in the heterocycle include B, N, O, S, Se and Te. The heteroatom in the heterocycle is particularly preferably N, O or S. A carbon atom in the heterocycle has preferably a free atomic valence (monovalent) (heterocyclic group is bound via this carbon atom). The number of carbon atom in the heterocyclic group is preferably 1 to 40, more preferably 1 to 30, still more preferably 1 to 20. Examples of the saturated heterocycle include a pyrrolidine ring, morpholine ring, 2-bora-1,3-dioxolane ring, and 1,3-thiazolidine ring. Examples of the unsaturated heterocycle include an imidazole ring, thiazole ring, benzothiazole ring, benzoxazole ring, benzotriazole ring, benzoselenazole ring, pyridine ring, pyrimidine ring and quinoline ring. The heterocyclic group may have substituents. Examples of such substituents are the same as those listed above as the “substituents of the alkyl moiety in the substituted alkyl group, substituted alkenyl group, substituted alkynyl group, and substituted aralkyl group”.

Next, the compound represented by formula (I) or (II) is described below. In formulae (I) and (II), R₁ represents a substituent. Examples of the substituent include the same examples as the substituent of the alkyl moiety of the above-described substituted alkyl group, substituted alkenyl group, substituted alkynyl group and substituted aralkyl group.

R₁ is preferably a halogen atom, an alkyl group, an alkenyl group, an alkynyl group, an aryl group, a cyano group, a hydroxyl group, a nitro group, a carboxyl group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group, an acylamino group, an aminocarbonylamino group, an alkoxycarbonylamino group, an aryloxycarbonylamino group, a sulfamoylamino group, an alkyl or aryl sulfonylamino group, a mercapto group, an alkylthio group, an arylthio group, a heterocyclic thio group, a sulfamoyl group, a sulfo group, an alkyl or aryl sulfinyl group, an alkyl or aryl sulfonyl group, an acyl group, an aryloxycarbonyl group, an alkoxycarbonyl group, a carbamoyl group, an imido group, a phosphino group, a phosphinyl group, a phosphinyloxy group, a phosphinylamino group, or a silyl group,

more preferably a halogen atom, an alkyl group, an aryl group, a cyano group, a hydroxyl group, a carboxyl group, an alkoxy group, an aryloxy group, a silyloxy group, a heterocyclic oxy group, an acyloxy group, a carbamoyloxy group, an amino group, an acylamino group, an aminocarbonylamino group, an alkoxycarbonylamino group, an aryloxycarbonylamino group, a sulfamoylamino group, an alkyl or aryl sulfonylamino group, a mercapto group, an alkylthio group, an arylthio group, a heterocyclic thio group, a sulfamoyl group, a sulfo group, an alkyl or aryl sulfinyl group, an alkyl or aryl sulfonyl group, a carbamoyl group, an imido group, a phosphino group, a phosphinyl group, a phosphinyloxy group, a phosphinylamino group, or a silyl group; still more preferably a halogen atom, an alkyl group, an aryl group, a hydroxyl group, an alkoxy group, an aryloxy group, an amino group, a mercapto group, an alkylthio group, an arylthio group, a sulfamoyl group, a sulfo group, an alkyl or aryl sulfinyl group, an alkyl or aryl sulfonyl group,
still furthermore preferably a halogen atom, an alkyl group, an aryl group, an alkoxy group, an aryloxy group, an alkylthio group, an arylthio group; still furthermore preferably a halogen atom, an alkyl group having 1 to 20 carbon atoms, an aryl group having 6 to 20 carbon atoms, an alkoxy group having 1 to 20 carbon atoms, an aryloxy group having 6 to 20 carbon atoms, an alkylthio group having 1 to 20 carbon atoms, an arylthio group having 6 to 20 carbon atoms; still furthermore preferably a chlorine atom, a fluorine atom, a bromine atom, an alkyl group having 1 to 8 carbon atoms, an aryl group having 6 to 10 carbon atoms, an alkoxy group having 1 to 8 carbon atoms, an aryloxy group having 6 to 10 carbon atoms, an alkylthio group having 1 to 8 carbon atoms, an arylthio group having 6 to 10 carbon atoms; still furthermore preferably a chlorine atom, a fluorine atom, an alkyl group having 1 to 4 carbon atoms and an alkoxy group having 1 to 4.

n₁ is preferably an integer of 0 to 3, further preferably an integer of 0 to 2, furthermore preferably an integer of 0 to 1, and most preferably 0. In other words, it is most preferable that a benzene ring does not have any substituent.

R₂ represents an n₂-valent substituent or a linking group. Examples of the substituent include the aforementioned examples of the substituent in the alkyl moiety of the substituted alkyl group, substituted alkenyl groups, substituted alkynyl groups and substituted aralkyl groups. Further, the linking group is a group in which the substituent additionally has one or more bonds.

R₂ is preferably an aliphatic group, an aromatic group, a heterocyclic group, or linking groups of these groups having additional bonds, more preferably an alkyl group, an alkenyl group, an alkynyl group, an aryl group, a heterocyclic group containing N, O or S as a hetero ring component and carbon atoms, or divalent to tetravalent linking groups of these groups, further preferably an alkyl group, an alkenyl group, an aryl group, a heterocyclic group containing N, O or S as a hetero ring component and carbon atoms, or divalent to trivalent linking groups of these groups, still further preferably an alkyl group having 1 to 20 carbon atoms, an alkenyl group having 2 to 20 carbon atoms, an aryl group having 6 to 20 carbon atoms, a 5- or 6-membered heterocyclic group containing N, O or S as a hetero ring component and carbon atoms, or divalent to trivalent linking groups of these groups, still further preferably an alkyl group having 1 to 8 carbon atoms, an alkenyl group having 2 to 8 carbon atoms, an aryl group having 6 to 12 carbon atoms, a 5- or 6-membered heterocyclic group containing N, O or S as a hetero ring component and carbon atoms, or divalent to trivalent linking groups of these groups, and still further preferably an alkyl group having 1 to 8 carbon atoms, an aryl group having 6 to 12 carbon atoms, a 5- or 6-membered heterocyclic group containing N, O or S as a hetero ring component and carbon atoms, or divalent to trivalent linking groups of these groups.

R₂ is more preferably methyl, ethyl, propyl, butyl, iso-propyl, 2-butyl, benzyl, phenyl, 2-naphthyl, pyrrol-2-yl, thiophen-2-yl, indol-1-yl, indol-2-yl, benzofuran-2-yl, benzothiophen-2-yl, ethylene, trimethylene, 1,2-propylene, tetramethylene, 1,2-phenylene, 1,3-phenylene, 1,4-phenylene, 2,6-naphthylene, pyrrol-2,5-yl, furan-2,5-yl, thiophen-2,5-yl, or benzene-1,3,5-yl; further preferably methyl, ethyl, benzyl, phenyl, pyrrol-2-yl, thiophen-2-yl, indol-1-yl, indol-2-yl, benzothiophen-2-yl, ethylene, trimethylene, 1,3-phenylene, 1,4-phenylene, pyrrol-2,5-yl, thiophen-2,5-yl, or benzene-1,3,5-yl; furthermore preferably ethylene, trimethylene, 1,3-phenylene, 1,4-phenylene, pyrrol-2,5-yl, thiophen-2,5-yl, or benzene-1,3,5-yl; and most preferably 1,4-phenylene.

n₂ is preferably an integer of 1 to 3, further preferably an integer of 2 to 3, and most preferably 2.

Hereinafter, the compound represented by the formula (I) in the present invention is exemplified, but the present invention is not limited thereto.

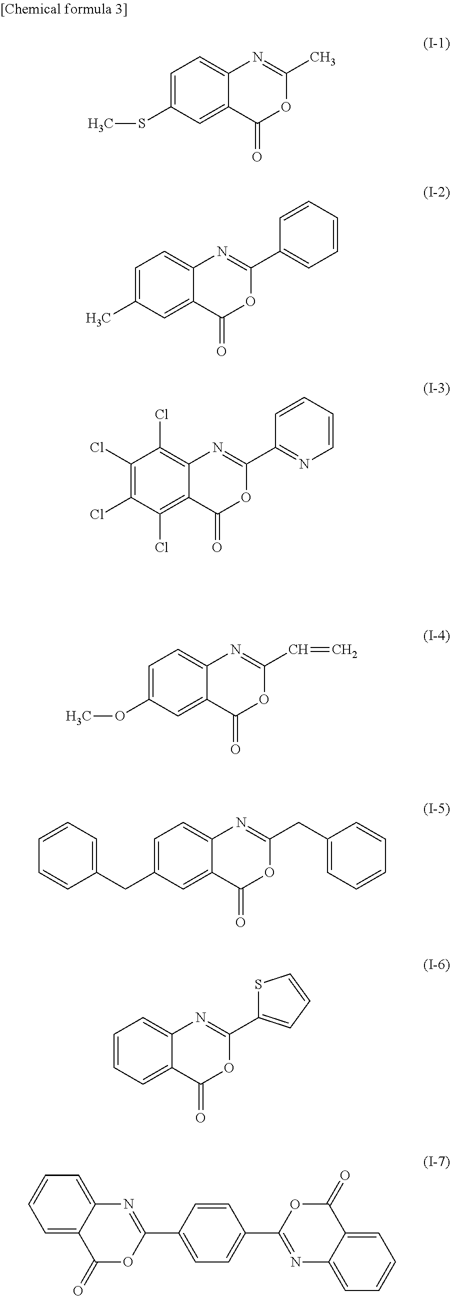

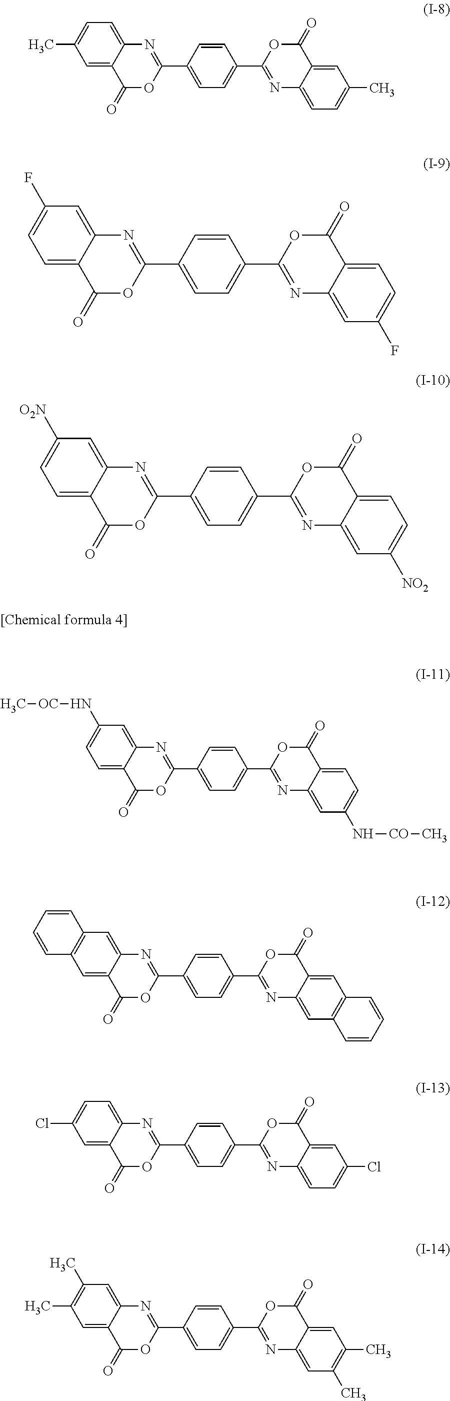

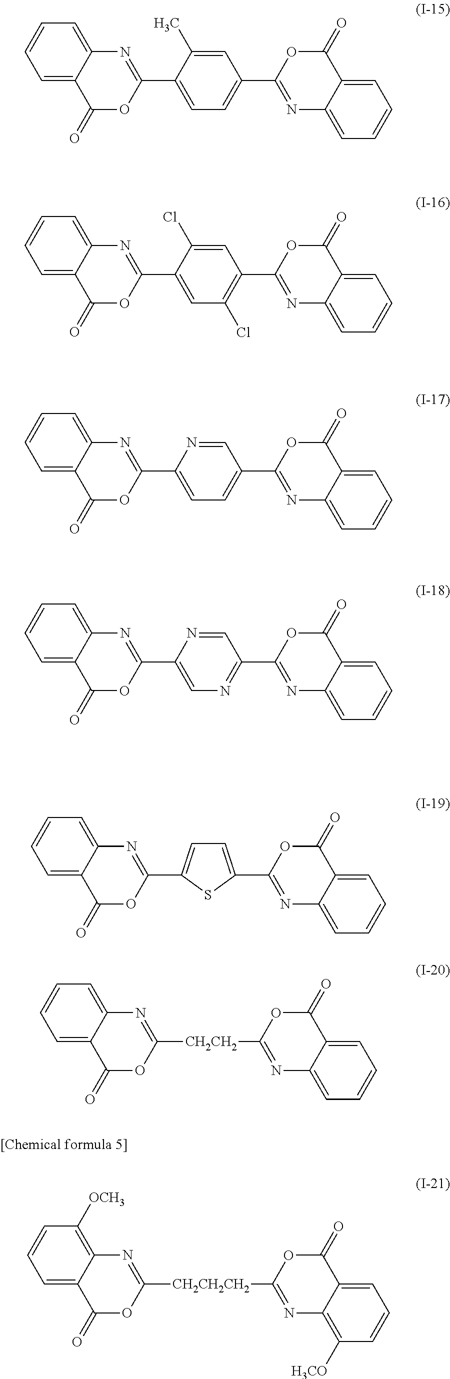

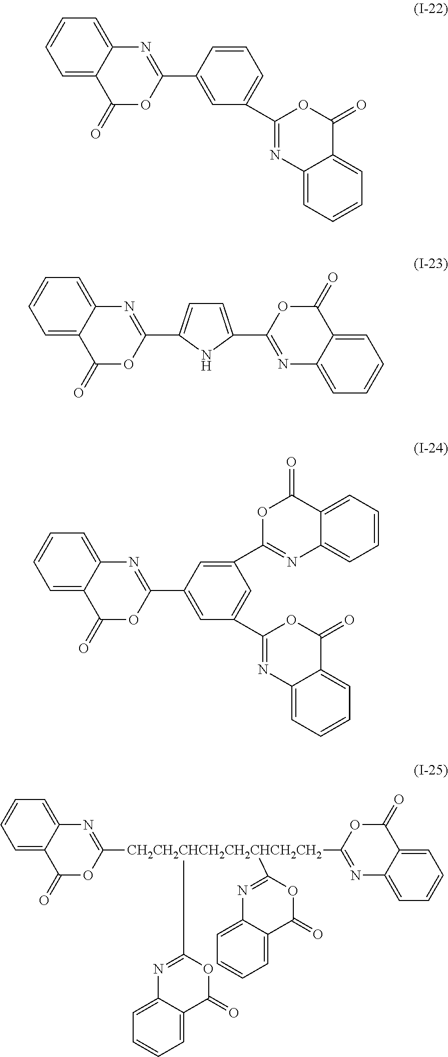

In the present invention, the method of producing the compound represented by the above-described formula (I) is not particularly limited, so long as an aluminum ion concentration is in the range of less than 2 ppm and further an iron ion concentration is in the range of less than 2 ppm. For example, it is possible to use synthetic methods such as a method described on page 7 of Japanese Patent No. 3874407, or a method described on page 3 of JP-A-58-194854 (“JP-A” means unexamined published Japanese patent application). A target compound can be obtained by arbitrarily carrying out operations such as recrystallization of a raw material (for example, purification), or recrystallization or sublimation-purification of the compound represented by formula (I). Alternatively, purification can be carried out by using isatoic anhydride as a starting material and further recrystallization, as described in JP-T-2005-507006 (“JP-T” means published Japanese translation of PCT application).

One of preferable embodiments of the present invention is an ultraviolet absorbent obtained by the following production method. The method of producing the compound represented by formula (I) according to the present invention includes a process A that an anthranilic acid compound is allowed to react with a carboxylic acid halide in the absence of a base. In the process A, an amide intermediate compound represented by the above-described formula (II) is synthesized. Further, a benzoxadione skeleton is formed in a process B that carries out dehydration and condensation of the amide intermediate compound represented by formula (II) produced in the process A, thereby producing the compound represented by formula (I).

Substituted or unsubstituted anthranilic acids may be used as the anthranilic acid compound that is used as a raw material. Examples of the substituted anthranilic acid include compounds in which hydrogen atom (s) on the benzene ring thereof is or are substituted with n₁ number (s) of substituent R₁ wherein R₁ represents a substituent and n₁ represents an integer of 0 to 4. R₁ and n₁ each have the same definitions as those of R₁ and n₁ in the above-described formula (I), and a preferable range of R₁ or n₁ is also the same as that of R₁ or n₁ in formula (I) respectively.

Carboxylic acid halide that is provided as a raw material is represented by R₂ (—COOX)n₂, wherein R₂ represents an n₂-valent substituent or linking group, and n₂ represents an integer of 0 to 4, and X represents a halogen atom. R₂ and n₂ each have the same definitions as those of R₂ and n₂ in the above-described formula (I), and a preferable range of R₂ or n₂ is also the same as that of R₂ or n₂ in formula (I) respectively.

As the ratio of raw materials used in the present reaction, the n₂-valent carboxylic acid halide is preferably used in a proportion of 0.3/n₂ to 2.0/n₂ mol, more preferably from 0.6/n₂ to 1.5/n₂ mol, and further preferably from 0.8/n₂ to 1.2/n₂ mol, relative to 1 mol of anthranilic acid compound respectively.

The reaction may be carried out in either absence or presence of solvent, with the presence of solvent being preferable. Examples of the solvent used in the presence of solvent include amide-series solvents (e.g., N,N-dimethylformamide, N,N-dimethylacetamide, and N-methyl-pyrrolidinone), sulfone-series solvents (e.g., sulfolane), ureido-series solvents (e.g., tetramethylurea), ether-series solvents (e.g., dioxane, and cyclopentyl methyl ether), ketone-series solvents (e.g., acetone, methyl ethyl ketone, and cyclohexanone), hydrocarbon-series solvents (e.g., toluene, xylene, and n-decane), halogen-series solvents (e.g., tetrachloroethane, and chlorobenzene), alcoholic solvents (e.g., methanol, ethanol, isopropyl alcohol, ethylene glycol, cyclohexanol, and phenol), ester-series solvents (e.g., ethyl acetate and butyl acetate), nitrile-series solvents (e.g., acetonitrile), and water. One or more such solvents may be used either singly or as combined. Further, it is also preferable to add supplementarily the same solvent or another solvent from those used in the process A, in the process B after completion of the process A. Further, it is preferable to use an aprotic solvent in the process A.

Further, through the process A and process B, the solvent having a donor number of 10 or more is preferably used. Details of the donor number of the solvent is described in, for example, “The donor-acceptor approach to molecular interactions” (Original title in English) authored by V. Gutmann and translated by Hitoshi Otaki and Isao Okada, pp. 21 to 29 (1983) (edited by Gakkai Shuppan Center). In the present invention, the donor number of the solvent is not limited to the values described in the above-described book or the like, but it is a matter of course that even in the case where the donor number is not known by literatures, if the donor number obtained by measurement according to a measuring method described in the literatures is fallen in the specified range, such measured values are also encompassed.

The donor number of the solvent is more preferably 15 or more, further preferably 20 or more, and still further preferably 25 or more. Examples of the solvent having donor number of 25 or more that is preferably used in the present invention include N,N-dimethylformamide, N,N-dimethylacetamide, N-methylpyrrolidinone, and hexamethylphosphoric acid triamide. Among these solvents, N,N-dimethylformamide, N,N-dimethylacetamide, and N-methylpyrrolidinone are more preferable.

The reaction temperature in the process A is ordinarily from −50 to 100° C., preferably from −40 to 70° C., further preferably from −30 to 50° C., still further preferably from −20 to 30° C., still further preferably from −15 to 20° C., still further preferably from −10 to 10° C., and especially preferably from 0 to 10° C.

On the other hand, the reaction temperature in the process B is ordinarily from 0 to 200° C., preferably from 30 to 180° C., further preferably from 50 to 150° C., and especially preferably from 80 to 130° C.

In the process B, it is preferable that at least one dehydration condensation agent exists together. Examples of preferable dehydration condensation agent include inorganic dehydration condensation agents (for example, acid anhydrides such as sulfur trioxide, or diphosphorous pentoxide; acid chlorides such as thionyl chloride, or phosphorous oxychloride); organic dehydration condensation agents (for example, acid anhydrides such as acetic acid anhydride, or propionic acid anhydride; acid halides such as acetyl chloride; N,N-dicyclohexylcarbodiimide); absorbents such as molecular sieves; and inorganic compounds that takes therein water as a crystal solvent, such as anhydrous sodium sulfate. Among these materials, inorganic or organic dehydration condensation agents are especially preferable. Inorganic or organic acid anhydrides are more preferable. Organic acid anhydrides are further preferable. Acetic acid anhydride is most preferable.

The maximum absorption wavelength of the ultraviolet absorbent of the present invention is not particularly limited, but preferably in the range of 300 to 390 nm, and more preferably from 335 to 355 nm.

The ultraviolet absorbent of the present invention has a low content of metal ions. Accordingly, the ultraviolet absorbent, when added to a thermoplastic polymer and kneaded, makes it possible to reduce deterioration of the thermoplastic polymer. A request level with respect to a high transparency of the optical lens and the like is increasing more than ever. Accordingly, the demand on improvement of transparency is increasing. The ultraviolet absorbent of the present invention is able to respond to this demand. Specifically, the ultraviolet absorbent of the present invention has an aluminum ion concentration of less than 2 ppm and an iron ion concentration of less than 2 ppm. The aluminum ion concentration is preferably less than 1 ppm, and more preferably less than 0.5 ppm. The iron ion concentration is preferably less than 1 ppm, and more preferably less than 0.5 ppm. Further, the calcium ion concentration is preferably less than 1 ppm. It is thought that the calcium ion concentration contributes only to reduction in intrinsic concentration variation that is caused by decomposition of the polymer.

In order to control the content of metal ions to a low level, it is preferable that the pH of the system at the time of both reaction and crystallization is low. The pH is preferably 5 or less, more preferably 3 or less, and most preferably 1 or less.

Next, polymer compositions are explained. The polymer composition of the present invention contains the ultraviolet absorbent of the present invention and a polymer material (preferably thermoplastic polymers). The ultraviolet absorbent of the present invention makes it possible to reduce deterioration of the thermoplastic polymer by use of the ultraviolet absorbent kneaded in the thermoplastic polymer.

The thermoplastic polymer used in the present invention is not particularly limited. Examples of the thermoplastic polymer include thermoplastic polyesters such as polyethyleneterephthalate, polyethylenenaphthalate, or polybutyleneterephthalate; polycarbonates; styrene polymers such as polystyrene, styrene-acrylonitrile-butadiene copolymer, or high-impact polystyrene; acrylic polymers; amide polymers; polyphenyleneether; polyolefin such as polyethylene, or polypropylene; polyvinylchloride; polyoxymethylene; polyphenylene sulfide; lactic acid polymers; and arbitrary mixtures of these thermoplastic polymers. The ultraviolet absorbent of the present invention has a profound effect, among these thermoplastic polymers, on polyethyleneterephthalate, polycarbonates or acrylic polymers. Further, the ultraviolet absorbent of the present invention has the most effect on polyethyleneterephthalate or polycarbonates.

The shape of the polymer material containing the ultraviolet absorbent according to the present invention may be flat film, powder, spherical particle, crushed particle, bulky continuous particle, fiber, tube, hollow fiber, granule, plate, porous particle, or the other.

The ultraviolet absorbent of the present invention may be contained in a polymer composition in an arbitrary quantity necessary to provide desired properties. If the content of the ultraviolet absorbent is too small, a sufficient ultraviolet-shielding effect can not be obtained. On the other hand, if the content thereof is excessively high, a problem of bleed-out arises. Though an adequate content varies depending on ultraviolet absorbing compounds and/or polymer materials used, one skilled in the art is able to determine such adequate content by experiment. The content is preferably in the range of from more than 0% by mass to 20% by mass, more preferably from more than 0% by mass to 10% by mass, and further preferably from 0.05% by mass to 5% by mass, based on the polymer composition respectively.

The polymer material containing the ultraviolet absorbent according to the present invention may contain any additives such as antioxidant, photostabilizer, processing stabilizer, antidegradant, and compatibilizer, as needed in addition to the polymer substance above and the ultraviolet absorbent according to the present invention.

### EXAMPLES

The present invention will be described in more detail based on the following examples, but the invention is not intended to be limited thereby.

### Example 1

### Preparation of Exemplified Compound (I-7)

120.7 g of anthranilic acid and 1000 ml of N-methylpyrrolidinone were placed in a three-necked flask, and were dissolved while stirring. Stirring of the resultant solution was continued while ice cooling. To the solution, 89.3 g of terephthalic acid dichloride was added and stirred without change for 2 hours. At this time, an internal temperature was in the range of 3 to 8° C. Thereafter, 225 g of acetic acid anhydride and 500 ml of N-methylpyrrolidinone were added, and then the temperature was elevated. The resultant mixture was heated at an internal temperature ranging from 108 to 116° C. for 2 hours while stirring. Then, crystals obtained by cooling at 30° C. or lower were collected by filtration and dried. As a result, 155.6 g of exemplified compound (I-7) was obtained as a target compound (96% in yield).

The melting point of the exemplified compound (I-7) obtained in the present Example is shown in the following Table 1.

Further, 10 g of the exemplified compound (I-7) obtained in the present Example was precisely weighed in a crucible and heated at 700° C. for 6 hours to ash. After ashing, 1 ml of nitric acid was added to the sample, and the sample was dissolved. Thereafter, the resultant solution was diluted with ultrapure water so that the total amount was 100 ml. A content of metal ions in the solution was measured using ICP Atomic Emission Spectrometer ICPS-7000 (trade name, manufactured by Shimadzu Corporation). The results are shown in the following Table 1.

Further, the maximum absorption wavelength (λmax) in a toluene solution (2.3×10⁻⁵ mol/liter) of the exemplified compound (I-7) obtained in the present Example was measured using U-4100 Type spectrophotometer (trade name, manufactured by Hitachi, Ltd.). The result is shown in the following Table 1.

### Example 2

### Preparation of Exemplified Compound (I-7)

120.7 g of anthranilic acid and 1000 ml of N,N-dimethylacetamide were placed in a three-necked flask, and were dissolved while stirring. Stirring of the resultant solution was continued while ice cooling. To the solution, 89.3 g of terephthalic acid dichloride was added and stirred without change for 1 hour. During the time, an internal temperature was in the range of 0 to 5° C. Thereafter, 225 g of acetic acid anhydride and 500 ml of toluene were added, and then the temperature was elevated. The resultant mixture was heated under reflux of the solvent for 1.5 hours while stirring. Then, crystals obtained by cooling to 30° C. or lower were collected by filtration and dried. As a result, 160.5 g of exemplified compound (I-7) was obtained as a target compound (99% in yield).

The melting point, the content of metal ions, and the maximum absorption wavelength (λmax) in a toluene solution of the exemplified compound (I-7) obtained in the present Example were measured in the same manner as in Example 1. The results are shown in the following Table 1.

### Comparative Example 1

### Preparation of Exemplified Compound (I-7)

120.7 g of anthranilic acid, 45.7 g of anhydrous sodium carbonate and 880 ml of water were placed in a three-necked flask, and were dissolved while stirring. To the resultant solution, a solution of 89.8 g of terephthalic acid dichloride dissolved in 2700 ml of acetone was dropped using a dropping funnel at room temperature, and then amidation reaction between anthranilic acid and terephthalic acid dichloride was carried out under reflux for 1 hour. Thus, a slurry of solid containing N,N′-bis(o-carboxyphenylterephthalamide) was obtained. The solid was separated by filtration from the slurry, and washed with 2700 ml of water and then dried. As a result, 175.6 g of the solid was obtained.

Next, 175.6 g of the dried solid, 899 g of acetic acid anhydride and 880 ml of toluene were placed in a 4-necked flask, and iminoesterification reaction was carried out under reflux for 6 hours. After cooling down to room temperature, a newly produced solid was collected by filtration. The collected solid was washed with 880 ml of acetone, and then dried. As a result, 155.3 g of solid containing exemplified compound (I-7) was obtained.

Finally, 155 g of the solid thus obtained and 600 g of water were placed in a flask, and 24.6 g of a 1% aqueous solution of sodium hydroxide was added while stirring. Further, stirring was continued at 25° C. for 30 minutes to complete an alkali treatment. The alkali-treated solid was collected by filtration and then washed with 1400 g of a 60° C. hot water. The washed solid was dehydrated and then dried using a 100° C. hot blow dryer for 2 hours. As a result, 146.4 g of exemplified compound (I-7) was obtained as a target compound (90% in yield).

The melting point, the content of metal ions, and the maximum absorption wavelength (λmax) in a toluene solution of the exemplified compound (I-7) obtained in the present Example were measured in the same manner as in Example 1. The results are shown in the following Table 1.

### Comparative Example 2

### Preparation of Exemplified Compound (I-7)

142.5 g of isatoic acid anhydride was dissolved in 1450 g of dry pyridine at 60° C. in a 3-necked flask. To the resultant mixture, 89.8 g of terephthalic acid dichloride was gradually added, while stirring and slightly cooling to keep the temperature constant. Next, the mixture was refluxed by heating for four hours. Then, the reactant was cooled to room temperature to obtain slurry. A newly produced solid was collected by filtration and then dried. As a result, 149.7 g of exemplified compound (I-7) was obtained as a target compound (92% in yield).

The melting point, the content of metal ions, and the maximum absorption wavelength (λmax) in a toluene solution of the exemplified compound (I-7) obtained in the present Example were measured in the same manner as in Example 1. The results are shown in the following Table 1.

TABLE 1

| | | | | | | Maximum |
|---|---|---|---|---|---|---|
| | Content of metal ions | | | | Melting | absorption |
| | (ppm) | | | | point | wavelength |
| | Al | Fe | Ca | Na | (° C.) | in solution |
| Example 1 | 0.3 | 0.4 | 0.3 | <0.1 | 317.3 | 349.5 |
| Example 2 | 0.3 | 0.3 | 0.3 | <0.1 | 316.3 | 349.5 |
| Comparative | 2.5 | 2.4 | 5 | <0.1 | 314.3 | 349.5 |
| example 1 | | | | | | |
| Comparative | 2.5 | 2.7 | 7.3 | <0.1 | 315.3 | 349.5 |
| example 2 | | | | | | |

### Example 3

### Production of Master Batch Pellet

12 parts by mass of the compound obtained in Example 1, which was dried, and 88 parts by mass of polyethyleneterephthalate resin (product of Mitsui Chemicals, Inc.) were mixed and a master batch pellet of the resultant mixture was produced using a kneading extruder. The extruding temperature was 285° C. and the extruding time was 8 minutes.

### Example 4

A master batch pellet was produced in the same manner as in Example 3, except that the compound of Example 2, which was dried, was used.

### Comparative Example 3

A master batch pellet was produced in the same manner as in Example 3, except that the compound of Comparative Example 1, which was dried, was used.

### Comparative Example 4

A master batch pellet was produced in the same manner as in Example 3, except that the compound of Comparative Example 2, which was dried, was used.

### <Evaluation of Master Batch Pellet>

The following evaluations were performed with respect to each of the produced master batch pellets. The results are shown in Table 2.

### (A) Intrinsic Viscosity of Polymer

An intrinsic viscosity of the polymer was measured at 25° C. using an Ostwald viscometer. In this measurement, o-chlorophenol was used as a solvent.

### (B) Evaluation of Yellow Index (YI)

A 1.5 mm thick injection plate was formed from each of the thus-produced master batch pellets. The YI value of the injection plate was measured.

### (C) Thermal Stability of Polymer

The master batch pellet was subjected to a heat treatment at 280° C. for 60 minutes in nitrogen atmosphere. An intrinsic viscosity after heat treatment was measured. The obtained value was designated as Δ IV. Further, a 1.5 mm thick injection plate was formed from the said master batch pellet. The YI value of the injection plate was measured to obtain Δ YI.

TABLE 2

Properties of kneaded polymer

| | Intrinsic | | | |
|---|---|---|---|---|
| | viscosity | ΔIV | YI | ΔYI |
| Example 3 | 0.600 | 0.09 | 35 | 5 |
| Example 4 | 0.600 | 0.1 | 35 | 6 |
| Comparative example 3 | 0.580 | 0.25 | 37 | 15 |
| Comparative example 4 | 0.590 | 0.25 | 38 | 20 |

As is apparent from the results shown in Table 2, it is understood that the master batch pellets of Examples 3 and 4 are more suppressed in terms of both reduction in intrinsic viscosity and increase in YI value over time by heating, whereby deterioration of polyester can be more suppressed, than those of comparative Examples 3 and 4.

### INDUSTRIAL APPLICABILITY

The benzoxadinone-based ultraviolet absorbent of the present invention makes it possible to reduce deterioration of a thermoplastic polymer when used in the form of the ultraviolet absorbent kneaded in the thermoplastic polymer. Further, according to the method of the present invention, it is possible to produce a high-quality benzoxadinone-based ultraviolet absorbent having a low content of metal ions.

Having described our invention as related to the present embodiments, it is our intention that the present invention not be limited by any of the details of the description, unless otherwise specified, but rather be construed broadly within its spirit and scope as set out in the accompanying claims.

This application claims priority on Patent Application No. 2008-091833 filed in Japan on Mar. 31, 2008, which is entirely herein incorporated by reference.

1. An ultraviolet absorbent comprising a compound represented by the following formula (I), having an aluminum ion in a concentration of less than 2 ppm, and an iron ion in a concentration of less than 2 ppm. 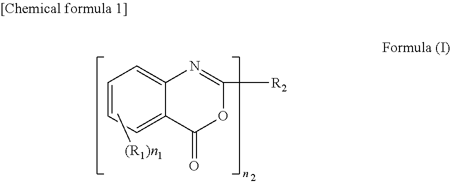
wherein R₁ represents a substituent; n₁ represents an integer of 0 to 4; R₂ represents an n₂-valent substituent or a linking group; and n₂ represents an integer of 1 to 4.

2. The ultraviolet absorbent according to claim 1, wherein the aluminum ion concentration is less than 1 ppm, and the iron ion concentration is less than 1 ppm.

3. The ultraviolet absorbent according to claim 1, wherein the aluminum ion concentration is less than 0.5 ppm, and the iron ion concentration is less than 0.5 ppm.

4. The ultraviolet absorbent according to claim 1, further having a calcium ion in a concentration of less than 1 ppm.

5. A method of producing the ultraviolet absorbent according to claim 1, comprising:
a process A in which an anthranilic acid compound is allowed to react with a carboxylic acid halide in the absence of a base, without isolating an amide intermediate compound represented by the following formula (II). 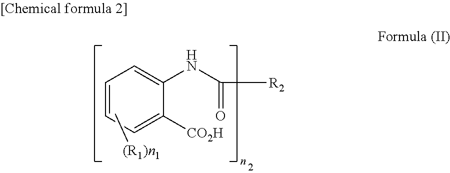
wherein R₁ represents a substituent; n₁ represents an integer of 0 to 4; R₂ represents an n₂-valent substituent or a linking group; and n₂ represents an integer of 1 to 4.

6. The method according to claim 5, wherein at least one of reaction solvents used in the process A has a number of donor of 10 or more.

7. The method according to claim 5, wherein no protic solvent is used in the process A.

8. The method according to claim 5, wherein the temperature at the process A is 50° C. or lower.

9. A polymer composition, comprising the ultraviolet absorbent according to claim 1, and a polymer substance.

10. The polymer composition according to claim 9, wherein the polymer composition is a film.

11. The polymer composition according to claim 9, wherein the polymer substance is a polyester.

12. The polymer composition according to claim 9, wherein the polymer substance is a polyethylene terephthalate.
